(19) United States
(12) Patent Application Publication
(10) Pub. No.: US 2021/0137970 A1
(43) Pub. Date: May 13, 2021

(54) NITRIC OXIDE-RELEASING PARTICLES

(71) Applicant: Diabetic Health, Inc., Durham, NC (US)

(72) Inventors: Wesley Langdon Storm, Durham, NC (US); Jonathan Edward McDunn, Cary, NC (US)

(21) Appl. No.: 17/090,637

(22) Filed: Nov. 5, 2020

Related U.S. Application Data
(60) Provisional application No. 62/932,070, filed on Nov. 7, 2019.

(51) Int. Cl. A61K 33/00; A61K 9/16; A61L 31/16; A61L 31/10; A61L 31/12; A61L 31/06; C08K 9/04; C08K 5/22; C08K 3/36

(52) U.S. Cl. CPC A61K 33/00; A61K 9/1611; A61L 31/16; A61L 31/10; A61L 2300/114; A61L 31/06; C08K 9/04; C08K 5/22; C08K 3/36; A61L 31/128

(57) ABSTRACT

Methods of preparing nitric oxide-releasing silica particles are provided, including the steps of functionalizing a silica particle with a linker moiety; and reacting a functional group on a nitric oxide storage/release compound with the linker moiety to attach the nitric oxide storage/release compound to the silica particle via the linker moiety. The disclosure also provides certain nitric oxide-releasing silica particles and precursors thereof, as well as compositions containing such particles.

### CROSS-REFERENCE TO RELATED APPLICATIONS

The present application claims priority to U.S. Provisional Patent Application No. 62/932,070, filed Nov. 7, 2019, which is incorporated herein by reference in its entirety.

### FIELD OF THE INVENTION

The present invention relates to functionalized particles (e.g., functionalized silica particles) and methods of making and using the same.

### BACKGROUND OF THE INVENTION

Nitric oxide (NO) is an endogenous, physiologically active metabolite. Within the human body, nitric oxide is produced, e.g., by oxidation of the guanidine group of L-arginine, facilitated by various nitric oxide synthase (NOS) enzymes. Human NOS enzymes include endothelial (eNOS or NOS-1) enzymes, inducible (iNOS or NOS-2) enzymes, and neuronal (nNOS or NOS-3) enzymes. Based on differential tissue expression and regulation of these enzymes, the NO produced within the body regulates diverse physiologic processes, including, but not limited to, vascular tone, immunologic response to inflammatory and infectious stimuli, and neurophysiology. Wound healing, vasodilation, angiogenesis, platelet aggregation, long-term memory potentiation, and inflammation all depend on NO in its role as a gasotransmitter to initiate and mediate these processes.

Based, in part, on these numerous functions within the body, NO shows promise for treating various diseases, infections, and conditions in the body. For example, NO possesses broad antimicrobial activity and thus can be effective as a therapeutic for serious skin and soft tissue infections. NO is a gas, which is not conveniently employed to treat such diseases, infections, and conditions. As such, certain research efforts have focused on providing NO-releasing compounds and materials to deliver and release NO at the desired site within the body for treatment. In particular, NO-releasing materials may be useful when employed in association with, e.g., devices implanted within the body (reducing inflammation associated with the implantation process, inhibiting bacterial growth, or reducing the foreign body response).

Manufacturing nitric oxide-releasing materials, such as particles, e.g., nanoparticles, for biomedical and other commercial and research applications has numerous challenges related to process controls and product quality. It would be useful to provide novel materials and methods to provide materials suitable for efficient NO storage/release.

### SUMMARY OF THE INVENTION

The present disclosure provides a multi-step process for preparing nitric oxide releasing materials and, in particular, nitric oxide-releasing particles. This process separates particle preparation from the synthesis of NO-storage/release moieties and these molecules are covalently attached in a later step through the use of a hetero(bi/poly/multi)functional linker. Following this type of process, the particles can be prepared and assessed independently from the NO-storage/release moiety. The hetero(bi/poly/multi)functional linker can be attached to the particle and the NO-storage/release moiety can then be covalently attached to the remaining reactive site(s) on the hetero(bi/poly/multi)functional linker. In some embodiments, bond formation between the NO-storage/release moiety and the hetero(bi/poly/multi)functional linker generates a stoichiometric amount of a new functional group on the resultant particle. This functional group could be useful in a variety of ways, including in reporter assays to determine the amount of NO-storage/release material bound to a single particle or as a means to retain these particles within polymer matrices to facilitate coating of placed medical devices.

In one aspect, the present disclosure provides a process for preparing nitric oxide-releasing silica particles, comprising: functionalizing a silica particle with a linker moiety; and reacting the linker moiety with a functional group on a nitric oxide storage/release compound to attach the nitric oxide storage/release compound to the silica particle via the linker moiety. In certain embodiments, the silica particle is mesoporous.

In some embodiments, the linker moiety comprises an electrophilic functional group capable of reacting with the functional group on the nitric oxide storage/release compound. For example, the electrophilic functional group on the linker moiety can comprise, but is not limited to, an alkyl halide moiety, a carboxylic acid moiety, a thiolactone moiety, or a glycidoxy moiety.

In some embodiments, the reacting comprises reacting a diazenium diolate comprising at least one free primary amine with the linker moiety. In some embodiments, the reacting comprises the formation of an amide bond attaching the nitric oxide storage/release compound to the silica particle. In some embodiments, the reacting comprises reacting a compound selected from the group consisting of: a diethylenetriamine/nitric oxide adduct, e.g., 2-[2-azaniumylethyl-[hydroxy(nitroso)amino]amino]ethylazanium (“DETA/NO”); a sperminediazen-1-ium-1,2-diolate, e.g., (Z)-[3-aminopropyl-[4-(3-aminopropylamino)butyl]amino]-hydroxyimino-oxidoazanium (“SPERMI/NO”); an N-propylpropanediamine nitric oxide adduct, e.g., (E)-[3-aminopropyl(propyl)amino]-hydroxyimino-oxidoazanium (“PAPA/NO”); and hydroxy-hydroxyimino-[methyl-[6-(methylazaniumyl)hexyl]amino]azanium (“MAHMA/NO”) with the linker moiety.

The disclosed process, in some embodiments, can include additional method steps before, simultaneously with, or after any of the steps referenced above. For example, in certain embodiments, the process further comprises protecting one or more components of the nitric oxide storage/release compound prior to reacting the desired functional group on the nitric oxide storage/release compound with the linker moiety; and deprotecting the one or more components after the reacting. In certain embodiments, the process further comprises introducing an anchor moiety on the silica particle for retention of the nitric oxide-releasing silica particle within a matrix to which the nitric oxide-releasing silica particle may subsequently be added.

In certain embodiments, the functionalizing and reacting are done substantially simultaneously. In certain embodiments, the functionalizing and reacting are done sequentially.

The disclosure further provides a process for providing a biocompatible coating or membrane, comprising: preparing nitric oxide-releasing silica particles according to the process referenced herein above; suspending the nitric oxide-releasing silica particles in a medium to give a functionalized medium; and processing the functionalized medium to provide a biocompatible coating or membrane.

In addition, the disclosure provides a process for improving one or more of functional, structural, mechanical, or electrical properties of a surface of a medical device, comprising: preparing nitric oxide-releasing silica particles according to the process of any of claims 1-11; suspending the nitric oxide-releasing silica particles in a medium to give a functionalized medium; and applying the functionalized medium to at least a portion of the surface of the medical device. In certain embodiments, the medium comprises a polymer.

The disclosure further provides a process for preparing a nitric oxide-releasing silica particle precursor, comprising: functionalizing a silica particle with a linker moiety comprising a thiolactone.

In another aspect, the disclosure provides a nitric oxide-releasing silica particle, comprising: a silica particle; a linker moiety comprising a thiolactone, wherein the linker moiety is attached to the silica particle; and a nitric oxide storage/release compound attached to the linking moiety. In some embodiments, the nitric oxide storage/release compound attached to the linking moiety comprises a polyamine backbone with at least one N-diazenium diolate and at least one nucleophilic primary amine.

The disclosure further provides a nitric oxide-releasing silica particle precursor, comprising: a silica particle; and a linker moiety comprising a thiolactone.

### BRIEF DESCRIPTION OF THE DRAWING

In order to provide an understanding of embodiments of the invention, reference is made to the appended drawing, which is not necessarily drawn to scale, and in which reference numerals refer to components of exemplary embodiments of the invention. The drawing is exemplary only, and should not be construed as limiting the invention.

FIG. 1 is a schematic depiction of a process of the present disclosure for the preparation of a functionalized particle.

### DETAILED DESCRIPTION OF THE INVENTION

The present invention now will be described more fully hereinafter. This invention may, however, be embodied in many different forms and should not be construed as limited to the embodiments set forth herein; rather, these embodiments are provided so that this disclosure will be thorough and complete, and will fully convey the scope of the invention to those skilled in the art. As used in this specification and the claims, the singular forms “a,” “an,” and “the” include plural referents unless the context clearly dictates otherwise. Reference to “dry weight percent” or “dry weight basis” refers to weight on the basis of dry ingredients (i.e., all ingredients except water).

The present disclosure generally provides a method for the production of functionalized particles and describes intermediates and functionalized particles provided thereby. Preferably, the particle is at least partially formed of silica, and the disclosed method provides a nitric oxide (NO) storage/release-functionalized particle.

As shown in FIG. 1, the process generally comprises combining an NO storage/release precursor with a particle that is or has been modified with one or more linker precursors. By “NO storage/release precursor” is meant a reactant which, upon reaction with the linker precursor, becomes associated with (e.g., attached to) the particle via the linker and which is then capable of functioning as a nitric oxide donor (referred to as an NO storage/release component). However, it is to be noted that “NO storage/release precursors” may, themselves, exhibit NO storage/release properties. Similarly, “linker precursor” is used to refer to a moiety present on the particle, which upon reaction with the NO storage/release precursor, forms a “linker” between the NO storage/release component and the particle.

As such, the term “precursor” in the context of the NO storage/release precursor is intended only to differentiate between the NO storage/release reactant (which includes a functional group suitable for reaction with a moiety on the functionalized linker) and the NO storage/release component in its final form, e.g., attached to the linker, and to differentiate between the linker reactants on the silica particle (which include a functional group suitable for reaction with a moiety on the NO storage/release precursor) and the linker in its final form, e.g., attached to both the particle and the NO storage/release component. It is understood in the art generally that reactions between two reactants commonly result in the modification of one or more moieties on one or both reactants (forming a bond there between). As such, associating the NO storage/release precursor with the linker precursor according to the disclosed methods may, in some embodiments, result in the modification of at least a portion of the NO storage/release precursor molecule and/or the linker precursor on the particle. According to the present disclosure, therefore, the reactants are referred to as the “linker precursor” and the “NO storage/release precursor” and the resulting functionalized particle is described as comprising a “linker” and an “NO storage/release component.”

The types of particles to which the disclosed method is applicable can vary. In certain embodiments, particles employed according to the presently disclosed method may be about 100 to about 500 nm in diameter, about 100 to about 1000 nm in diameter, or about 1000 to about 5000 nm in diameter. In some embodiments, the particles are nanoparticles. By “nanoparticle” is meant a particle with a diameter of about 1 to about 100 nm. Particles are generally substantially spherical in shape but, in some embodiments, can vary somewhat from perfectly spherical. The disclosed method can be used in the context of samples of particle sizes with varying particle size distributions (e.g., the method is relevant for functionalization of particles both in samples where all particles are of similar sizes and in samples where particles have varying sizes).

Typically, the particles employed in the disclosed methods comprise silica and, in some embodiments, consist essentially of silica. In some embodiments, the silica particles are described as mesoporous, which is a term understood in the art to refer to materials having pore size diameters of about 2 to about 50 nm. Silica nanoparticles are known and referenced in the art, e.g., in Zhou et al., “Mesoporous Silica Nanoparticles for Drug and Gene Delivery,” Acta Pharmaceutica Sinica B. 8(2): 2018, pp. 165-177; Bharti et al., “Mesoporous Silica Nanoparticles in Target Drug Delivery System: A Review,” Int. J. Pharm. Investigation 5(3): 2015, pp. 124-133, Lodha et al., “Synthesis of Mesoporous Silica Nanoparticles and Drug Loading of Poorly Water Soluble Drug Cyclosporin A,” J. Pharm. Bioallied Sci. 4 (Supp. 1): 2012, pp. S92-S94, which are incorporated herein by reference in their entireties. In certain embodiments, the particles may expressly exclude the presence of any material other than silica.

Two common types of mesoporous silica particles which may, in some embodiments, be functionalized according to the disclosed methods, are MCM-41 and SBA-15; however, the disclosure is not limited thereto. Further specific types of mesoporous silica particles that can be used include, but are not limited to, TUD-1, HMM-33, FSM-16, and combinations thereof.

These particles, according to the disclosed method, are functionalized with one or more “linker precursors.” The linker precursor is present on the particle to attach an NO storage/release component to the particle. Typically, the linker precursor is associated with the particle via a covalent bond, e.g., through a carboxylate group, glycidoxy group, thiolactone, or alkyl halide present on the surface of the particle. In some embodiments, the linker precursor is attached to the particle by means of a moiety comprising [dimethylamino(triazolo[4,5-b]pyridin-3-yloxy)methylidene]-dimethylazanium;hexafluorophosphate (“HATU”), [benzotriazol-1-yloxy(dimethylamino)methylidene]-dimethylazanium;hexafluorophosphate (“HBTU”), [(6-chlorobenzotriazol-1-yl)oxy-(dimethylamino)methylidene]-dimethylazanium;hexafluorophosphate (“HCTU”), [dimethylamino(triazolo[4,5-b]pyridin-3-yloxy)methylidene]-dimethylazanium;tetrafluoroborate (“TATU”), [benzotriazol-1-yloxy(dimethylamino)methylidene]-dimethylazanium;tetrafluoroborate (“TBTU”), [[(1-cyano-2-ethoxy-2-oxoethylidene)amino]oxy-(dimethylamino)methylidene]-dimethylazanium;tetrafluoroborate (“TOTU”), [[(Z)-(1-cyano-2-ethoxy-2-oxoethylidene)amino]oxy-morpholin-4-ylmethylidene]-dimethylazanium;hexafluorophosphate (“COMU”), [dimethylamino-[(4-oxo-1,2,3-benzotriazin-3-yl)oxy]methylidene]-dimethylazanium;tetrafluoroborate (“TDBTU”), [dimethylamino-(2,5-dioxopyrrolidin-1-yl)oxymethylidene]-dimethylazanium;tetrafluoroborate (“TSTU”), [dimethylamino-[(3,5-dioxo-4-azatricyclo[5.2.1.0^{2,6}]dec-8-en-4-yl)oxy]methylidene]-dimethylazanium;tetrafluoroborate (“TNTU”), [dimethylamino-(2-oxopyridin-1-yl)oxymethylidene]-dimethylazanium;tetrafluoroborate (“TPTU”), N-(3-dimethylaminopropyl)-N′-ethylcarbonate (“EDC”), N,N′-dicyclohexylcarbodiimide (“DCC”), 2,4,6-tripropyl-1,3,5,2λ⁵,4λ⁵,6λ⁵-trioxatriphosphinane 2,4,6-trioxide (“T3P”), (benzotriazol-1-yloxy-tris(dimethylamino)phosphonium hexafluorophosphate) (“BOP”), tripyrrolidin-1-yl(triazolo[4,5-b]pyridin-3-yloxy)phosphanium;hexafluorophosphate (“PyAOP”), benzotriazol-1-yloxy(tripyrrolidin-1-yl)phosphanium;hexafluorophosphate (“PyBOP”), bromo(tripyrrolidin-1-yl)phosphanium;hexafluorophosphate (“PyBrOP”), hexafluorophosphate (“PF₆”), or di(imidazol-1-yl)methanone (“CDI”).

Such linker precursors can vary and may, in some embodiments, be dependent upon the NO storage/release precursor to be associated with the silica particle in a subsequent step. In certain embodiments, the linker precursor comprises an electrophilic functional group capable of reacting with a moiety on the nitric oxide storage/release precursor. The electrophilic functional group can be, but is not limited to, a group comprising an alkyl halide moiety, a carboxylic acid moiety, a thiolactone moiety, or a glycidoxy moiety.

The term “alkyl” as used herein means saturated straight, branched, or cyclic hydrocarbon groups (i.e., cycloalkyl). In particular embodiments, alkyl refers to groups comprising 1 to 10 carbon atoms (“C1-10 alkyl”). In further embodiments, alkyl refers to groups comprising 1 to 8 carbon atoms (“C1-8 alkyl”), 1 to 6 carbon atoms (“C1-6 alkyl”), or 1 to 4 carbon atoms (“C1-4 alkyl”). In other embodiments, alkyl refers to groups comprising 3-10 carbon atoms (“C3-10 alkyl”), 3-8 carbon atoms (“C3-8 alkyl”), or 3-6 carbon atoms (“C3-6 alkyl”). In any of the foregoing, the alkyl group can be optionally substituted. In specific embodiments, alkyl refers to methyl, trifluoromethyl, ethyl, propyl, isopropyl, cyclopropyl, butyl, isobutyl, t-butyl, pentyl, cyclopentyl, isopentyl, neopentyl, hexyl, isohexyl, cyclohexyl, cyclohexylmethyl, 3-methylpentyl, 2,2-dimethybutyl, and 2,3-dimethylbutyl.

The term “halide” as used herein refers to Cl, F, Br, or I.

The term “carboxylic acid” refers to a compound comprising a group represented by C(═O)OH).

The term “thiolactone” refers to a sulfur-containing analogue of a lactone (which is understood to be a cyclic ester of a hydroxycarboxylic acids, containing a 1-oxacycloalkan-2-one structure), wherein the sulfur atom replaces one or more of the oxygens of the lactone.

The term “glycidoxy group” refers to an organic group comprising both a non-cyclic ether and cyclic ether having the general structure R′—O—CH₂—C₂H₄O.

Preparation of linker precursor-derivatized particles typically involves contacting the particle with a compound comprising the linker precursor, and comprising at least one moiety thereon suitable for reaction with a functional group present on the surface of the particle. In certain embodiments, the particle comprises carboxylate functionalities on its surface, and the particle is reacted with compounds comprising an amine to form an amide linkage between the particle and the linker precursor. Thus, such compounds used in the preparation of linker precursor-functionalized particles are typically bifunctional, i.e., having one portion suitable for attachment to the particle and one portion suitable for subsequent attachment of the NO storage/release precursor. Such reactions between a particle (e.g., a silica particle in some embodiments) and linker precursor can be conducted in various solvents and under various conditions, e.g., in anhydrous solvents including, but not limited to, toluene, at temperatures of 20-100° C.

The linker precursor-derivatized particle is reacted with the NO storage/release precursor to form a nitric oxide (NO) storage/release-functionalized particle. Again, the conditions of reaction can vary, e.g., the reaction can be conducted in various solvents and under various conditions, e.g., in anhydrous polar aprotic solvents, including, but not limited to, dimethylformamide (DMF) or dimethylsulfoxide (DMSO). Advantageously, the association of the NO storage/release precursor with the linker precursor-derivatized particle, in some embodiments, does not require elevated pressure and/or addition of base (which is employed in certain existing preparation methods, and which may destroy and/or damage at least some portion of the particles).

The NO storage release precursor comprises at least one functional group sufficient for reaction with at least one functional group present on the linker precursor, affording (upon reaction) attachment between the linker and the NO storage/release component. The composition of the NO storage/release precursor can vary, and may be any compound containing: a) a moiety capable of NO storage and/or release and b) a moiety capable of reaction with the linker precursor (which, upon reaction with the linker precursor, does not destroy the NO storage and/or release capability of the resulting product).

In some embodiments, the NO storage/release precursor comprises a polyamine backbone. In some embodiments, the NO storage/release precursor comprises a nucleophilic primary amine moiety. In some embodiments, the NO storage/release precursor comprises a diazenium diolate comprising at least one free primary amine, wherein the amine reacts with a moiety on the linker precursor to attach the NO storage/release component to the particle. Such reactions may, in some embodiments, result in the formation of an amide bond between the linker and the NO storage/release component. The NO storage/release precursor, in some embodiments, comprises one or more of a diethylenetriamine/nitric oxide adduct, e.g., 2-[2-azaniumylethyl-[hydroxy(nitroso)amino]amino]ethylazanium (DETA/NO), a sperminediazen-1-ium-1,2-diolate, e.g., (2)-[3-aminopropyl-[4-(3-aminopropylamino)butyl]amino]-hydroxyimino-oxidoazanium (SPERMI/NO), an N-propylpropanediamine nitric oxide adduct, e.g., (E)-[3-aminopropyl(propyl)amino]-hydroxyimino-oxidoazanium (PAPA/NO), and hydroxy-hydroxyimino-[methyl-[6-(methylazaniumyl)hexyl]amino]azanium (MAHMA/NO).

In reacting the NO storage/release precursor with the linker precursor-functionalized particle, it may be advantageous, in some embodiments, to protect one or more components of the NO storage release/precursor prior to reaction, and then to deprotect such components after reaction. Methods and reagents for protection and deprotection of various functionalities are generally known in the art, e.g., as described in Greene's Protective Groups in Organic Synthesis, P. Wuts and T. Greene, April 2006: John Wiley & Sons, Inc. which is incorporated herein by reference in its entirety.

Although the method described herein above assumes preparation of the linker precursor-functionalized particle first, followed by reaction of the linker precursor with the NO storage/release precursor, the disclosure is not limited thereto. In some embodiments, it may be possible to perform these steps substantially simultaneously, e.g., by combining the particle, the compound comprising the linker precursor, and the NO storage/release precursor so as to form the desired product. It is understood that such a process (combining all reactants together) is only suitable for certain combinations of reactants and its suitability may depend, for example, on appropriate functionalities/reactivities of the reactants and/or appropriate steric effects to ensure the reactants provide the desired product, comprising the particle connected to the NO release/storage component by means of the linker.

Various other functionalities can, in some embodiments, be introduced on the NO storage/release-functionalized particle. Such other functionalities can be associated with any portion of the functionalized particle, e.g., on the particle itself, on the linker, or on the NO storage/release component (without affecting the NO storage/release capability of the component). For example, in some embodiments, the method further comprises introducing an “anchor moiety” on the NO storage/release-functionalized particle. In preferred embodiments, such an anchor moiety is connected to the particle directly, e.g., by means of an alkyl halide, thiolactone, glycidoxy group, or carboxylate. By “anchor moiety” is meant a moiety that can provide for retention of the NO storage/release-functionalized particle with a matrix, e.g., including, but not limited to, a polymeric matrix. Exemplary anchor moieties include, but are not limited to, vinylic compounds, epoxy-containing compounds, thiols, terminal quaternary amines, and phosphate groups. One of skill in the art will recognize that appropriate consideration must be given to the functional groups associated with the reactants to ensure formation of the desired product.

Such additional functionalities can be introduced before, after, or substantially simultaneously with the other steps described herein. Although, in some embodiments, the “anchor moiety” is a separate moiety attached to the particle, it is noted that, in some embodiments, the anchor moiety can be formed as a consequence of the reaction between the linker precursor and the NO storage/release precursor. In such embodiments, the reaction resulting in the formation of the NO storage/release-functionalized particle may also provide an anchor moiety present on the functionalized particle.

The resulting NO storage/release-functionalized particles can be used in various ways and in various products. For example, in some embodiments, these functionalized particles are suspended in a medium (e.g., a polymeric medium) to give a functionalized biocompatible coating or membrane. In some embodiments, such a coating or membrane can be applied to a medical device, providing a functionalized coating on at least a portion of the surface thereof. The functionalized coating, in some embodiments, can improve one or more of functional, structural, mechanical, or electrical properties of the surface of the medical device upon which it is deposited.

Many modifications and other embodiments of the invention will come to mind to one skilled in the art to which this invention pertains having the benefit of the teachings presented in the foregoing description. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

What is claimed is:

1. A process for preparing nitric oxide-releasing silica particles, comprising:
functionalizing a silica particle with a linker moiety; and
reacting the linker moiety with a functional group on a nitric oxide storage/release compound to attach the nitric oxide storage/release compound to the silica particle via the linker moiety.

2. The process of claim 1, wherein the silica particle is mesoporous.

3. The process of claim 1, wherein the linker moiety comprises an electrophilic functional group capable of reacting with the functional group on the nitric oxide storage/release compound.

4. The process of claim 3, wherein the electrophilic functional group comprises an alkyl halide moiety, a carboxylic acid moiety, a thiolactone moiety, or a glycidoxy moiety.

5. The process of claim 1, wherein the reacting comprises reacting a diazenium diolate comprising at least one free primary amine with the linker moiety.

6. The process of claim 1, wherein the reacting comprises the formation of an amide bond attaching the nitric oxide storage/release compound to the silica particle.

7. The process of claim 1, wherein the reacting comprises reacting a compound selected from the group consisting of 2-[2-azaniumylethyl-[hydroxy(nitroso)amino]amino]ethylazanium (DETA/NO), (Z)-[3-aminopropyl-[4-(3-aminopropylamino)butyl]amino]-hydroxyimino-oxidoazanium (SPERMI/NO), (E)-[3-aminopropyl(propyl)amino]-hydroxyimino-oxidoazanium (PAPA/NO), and hydroxy-hydroxyimino-[methyl-[6-(methylazaniumyl)hexyl]amino]azanium (MAHMA/NO) with the linker moiety.

8. The process of claim 1, further comprising protecting one or more components of the nitric oxide storage/release compound prior to reacting the functional group on the nitric oxide storage/release compound with the linker moiety; and deprotecting the one or more components after the reacting.

9. The process of claim 1, wherein the functionalizing and reacting are done substantially simultaneously.

10. The process of claim 1, wherein the functionalizing and reacting are done sequentially.

11. The process of claim 1, further comprising introducing an anchor moiety on the silica particle for retention of the nitric oxide-releasing silica particle within a matrix to which the nitric oxide-releasing silica particle may subsequently be added.

12. A process for providing a biocompatible coating or membrane, comprising:
preparing nitric oxide-releasing silica particles according to the process of claim 1;
suspending the nitric oxide-releasing silica particles in a medium to give a functionalized medium; and
processing the functionalized medium to provide a biocompatible coating or membrane.

13. A process for improving one or more of functional, structural, mechanical, or electrical properties of a surface of a medical device, comprising:
preparing nitric oxide-releasing silica particles according to the process of claim 1;
suspending the nitric oxide-releasing silica particles in a medium to give a functionalized medium; and
applying the functionalized medium to at least a portion of the surface of the medical device.

14. The process of claim 13, wherein the medium comprises a polymer.

15. A process for preparing a nitric oxide-releasing silica particle precursor, comprising:
functionalizing a silica particle with a linker moiety comprising a thiolactone.

16. A nitric oxide-releasing silica particle, comprising:
a silica particle;
a linker moiety comprising a thiolactone, wherein the linker moiety is attached to the silica particle; and
a nitric oxide storage/release compound attached to the linking moiety.

17. The nitric oxide-releasing silica particle of claim 16, wherein the nitric oxide storage/release compound attached to the linking moiety comprises a polyamine backbone with at least one N-diazenium diolate and at least one nucleophilic primary amine.

18. A nitric oxide-releasing silica particle precursor, comprising:
a silica particle; and
a linker moiety comprising a thiolactone.
